(19) United States
(12) Patent Application Publication
(10) Pub. No.: US 2023/0158015 A1
(43) Pub. Date: May 25, 2023

(54) DRUG COMBINATIONS FOR INHIBITING INFLAMMATION AND SRC KINASE ACTIVATION FOLLOWING INVASIVE SURGICAL PROCEDURES

(71) Applicants: Gina E. VOTTA-VELLIS, Chicago, IL (US); SINTETICA S.A., Mendrisio (CH)

(72) Inventors: AUGUSTO MITIDIERI, Mendrisio (CH); ELISABETTA DONATI, Mendrisio (CH); ALAIN BORGEAT, Mendrisio (CH); E. GINA VOTTA-VELIS, Chicago, IL (US); MIRO VENTURI, Mendrisio (CH)

(21) Appl. No.: 17/802,956

(22) PCT Filed: Mar. 3, 2021
(86) PCT No.: PCT/US2021/020674

§ 371 Date: Aug. 28, 2022

Related U.S. Application Data
(60) Provisional application No. 62/985,962, filed on Mar. 6, 2020.

(51) Int. Cl. A61K 31/485; A61K 31/167; A61K 9/00; A61P 41/00; A61P 35/00; A61P 25/04; A61P 29/00; A61P 35/04; A61P 1/18

(52) U.S. Cl. CPC A61K 31/485; A61K 9/0019; A61K 31/167; A61P 1/18; A61P 25/04; A61P 29/00; A61P 35/00; A61P 35/04; A61P 41/00

(57) ABSTRACT

Combinations of compounds that inhibit activation of p-Src tyrosine kinase, having particularly utility in the treatment of inflammation resulting from traumatic surgical interventions and the proliferation or metastasis of cancer cells following surgical excision of cancerous tissue.

### FIELD OF THE INVENTION

The present invention relates to combinations of compounds that inhibit activation of p-Src tyrosine kinase and inflammation, particularly following invasive surgical procedures for cancer and other medical issues. In a particularly preferred aspect, the invention relates to combinations of lidocaine and methylnaltrexone and their pharmaceutically acceptable salts for the prevention or treatment of inflammation, cancer proliferation and cancer metastasis following invasive surgical procedures.

### BACKGROUND

Members of the Src family of kinases (SFKs) are non-receptor tyrosine kinases involved in numerous signal transduction pathways. The catalytic, SH3 and SH2 domains are attached to the membrane-anchoring SH4 domain through the intrinsically disordered “Unique” domains, which exhibit strong sequence divergence among SFK members. In the last two decades, structural and biochemical studies have begun to uncover the crucial role of the Unique domain in the regulation of SFK activity.

Src is a non-receptor protein tyrosine kinase with a key role in regulating cell-to-matrix adhesion, migration, and junctional stability (Frame, 2004 J. Cell Sci. 117(Pt 7), 989-998). Thus, precise regulation of Src activity is critical for normal cell growth. The inactive state of Src is obtained by phosphorylated tyrosine near the C-terminus of Src (Tyr530 in mammalian Src; Tyr527 in chicken Src), which is recognized by its SH2 domain, while the SH3 domain interacts with a polyproline motif located in the linker region between the SH2 and kinase domains; these intramolecular interactions restrict access to the kinase domain (Xu et al., 1997 Nature 385, 595-602). Dephosphorylation of Tyr530 is followed by autophosphorylation at Tyr419, leading to full activation of the kinase.

Potential roles in protein-protein interactions or cellular localization have been postulated for the phosphorylation of Src at Ser17 by PKA (cAMP-dependent protein kinase). For instance, it has been observed that the treatment of 3T3 fibroblasts with PDGF results in the translocation of Src from the plasma membrane to the cytosol, concomitant with an increase in phosphorylation of Ser17 by PKA (Walker et al., 1993 J. Biol. Chem. 268, 19552-19558). This observation suggests that this phosphorylation could interfere with the electrostatic interactions that act to anchor Src to the lipid bilayer. PKA phosphorylation of Src at Ser17 is also required in cAMP activation of Rap 1, inhibition of extracellular signal-regulated kinases, and inhibition of cell growth, although the mechanism by which this phosphorylation mediates these processes is not known (Obara et al., 2004 J. Cell Sci. 117, 6085-6094). See also Amata et al. (Frontiers in Genetics June 2014, Volume 5, Article 181, 1).

The peripheral µ-opioid receptor antagonist methylnaltrexone has been approved by the U.S. Food and Drug Administration and the European Medicines Agency since 2008 for the treatment of opioid-induced constipation in patients with advanced illness who are receiving palliative care when response to laxative therapy has not been sufficient, and most recently for opioid-induced constipation in patients with chronic pain. Because methylnaltrexone has restricted passage through the blood-brain barrier, it can be given to patients with cancer who are receiving opioid therapy without affecting analgesia.

In 2008, Singleton et al. (Mol Cancer Ther 2008;7(6). June 2008) reported that methylnaltrexone exerts a synergistic effect with 5-FU and bevacizumab on inhibition of vascular endothelial growth factor (VEGF)-induced human pulmonary microvascular endothelial cell proliferation and migration, two key components in cancer-associated angiogenesis. They also observed that treatment of human endothelial cells with methylnaltrexone, but not naltrexone, increased receptor protein tyrosine phosphatase activity, which was independent of µ-opioid receptor expression. These same researchers subsequently published several patent applications that proposed the use of methylnaltrexone to inhibit cellular proliferation and migration, particularly endothelial cell proliferation and migration associated with angiogenesis. See WO 2007/121447 by Moss et al.

In 2016, Janku et al. (Annals of Oncology 27: 2032-2038, 2016) explored pooled data from two randomized, placebo-controlled registration trials in patients with advanced disease and examined those with cancer to identify whether methylnaltrexone given at regular clinical doses could influence survival during the trial period. They concluded that treatment with methylnaltrexone was associated with increased overall survival, that this supported the preclinical hypothesis that µ-opioid receptor can play a role in cancer progression, and that targeting µ-opioid receptor with methylnaltrexone warrants further investigation in cancer therapy.

Lidocaine, 2-diethylaminoaceto-2′,6′-xylidide (C₁₄H₂₂N₂O), is an amide local anesthetic and a Class 1b antiarrhythmic agent according to the Vaughn Williams classification. A Class 1b antiarrhythmic agent binds to open sodium channels during phase 0 of the action potential, therefore blocking many of the channels when the action potential peaks. Approved indications for lignocaine include the requirement for local, neuraxial, regional or peripheral anesthesia by infiltration, block or topical application, or the prophylaxis or treatment of life-threatening ventricular arrhythmias. It has also been extensively used for chronic and neuropathic pain management, and more recently as an intravenous infusion for the management of postoperative analgesia and surgical recovery. Lidocaine has potential utility as a potent anti-inflammatory agent, although to date well-designed studies are lacking to substantiate its use in most clinical settings, and lidocaine is not approved for this specific indication. Weinberg et al., World J Anesthesial. Jul. 27, 2015, 4(2): 17-29.

As inflammatory processes involving Src tyrosine protein kinase and intercellular adhesion molecule-1 are important in tumor growth and metastasis, Piegeler et al. (Anesthesiology. 2012 September; 117(3): 548-559) hypothesized that amide linked local anesthetics such as lidocaine, chloroprocaine, and ropivacaine may inhibit inflammatory Src-signaling involved in migration of adenocarcinoma cells. To evaluate the effect of lidocaine on NCI-H838 lung cancer cell Src signaling, Piegeler et al. treated cells with increasing concentrations of lidocaine (1 nm, 1 µM, 10 µM, 100 µM) for 20 min and analyzed for Src phosphorylation via Western blot. Although a dose-dependent decrease in Src phosphorylation at tyrosine 419 was observed after incubation of the cells with lidocaine for 20 minutes, this decrease did not reach statistical significance (Kruskal-Wallis test, p=0.146). However, a significant decrease in TNF-α-induced Src phosphorylation of 73% was observed after co-incubation of cells with TNF-α and 10 µM (p=0.012) of lidocaine.

Work to date with inhibitors of Src-kinase phosphorylation has produced promising avenues for further research, but no real-world clinical data or treatments. What is needed are improved methods and compositions for preventing the activation of Src kinase.

Also needed are methods and compositions that have potential utility in a variety of medical conditions mediated by Src signalling, including inflammation, cellular proliferation and cellular migration involved in cancer angiogenesis and cancer metastasis.

### SUMMARY OF INVENTION

It has unexpectedly been discovered that lidocaine and methylnaltrexone act synergistically to inhibit the activation of Src protein kinase and inflammatory signalling, thus supporting the use of this combination in various conditions mediated by Src protein kinase activation and inflammation. Thus, in a first principal embodiment the invention provides a method of treating inflammation resulting from an invasive surgical procedure in a human in need thereof comprising administering to the human as an intravenous infusion: (a) a therapeutically effective amount of lidocaine or a pharmaceutically acceptable salt thereof; and (b) a therapeutically effective amount of methylnaltrexone or a pharmaceutically acceptable salt thereof.

The methods are also particularly useful for preventing the proliferation or spread of cancer after surgery on the cancer. Thus, in a second principal embodiment the invention provides a method of inhibiting proliferation or metastasis of cancer cells following surgical intervention to remove a cancerous tumor in a human in need thereof comprising administering to the human as an intravenous infusion: (a) a therapeutically effective amount of lidocaine or a pharmaceutically acceptable salt thereof; and (b) a therapeutically effective amount of methylnaltrexone or a pharmaceutically acceptable salt thereof.

The synergistic combination is particularly useful for suppressing inflammation or Src protein kinase activation after invasive surgical procedures. Thus, in a third principal embodiment the invention provides a method of inhibiting Src tyrosine protein kinase phosphorylation at Tyr419 following an invasive surgical procedure in a human in need thereof comprising administering to the human as an intravenous infusion: (a) a therapeutically effective amount of lidocaine or a pharmaceutically acceptable salt thereof; and (b) a therapeutically effective amount of methylnaltrexone or a pharmaceutically acceptable salt thereof.

In a fourth principal embodiment the invention provides a method of inhibiting cell signalling mediated by Src tyrosine protein kinase phosphorylation following an invasive surgical procedure in a human in need thereof comprising administering to the human as an intravenous infusion: (a) a therapeutically effective amount of lidocaine or a pharmaceutically acceptable salt thereof; and (b) a therapeutically effective amount of methylnaltrexone or a pharmaceutically acceptable salt thereof.

In a fifth principal embodiment the invention provides a method of treating a disease mediated by Src tyrosine protein kinase phosphorylation following an invasive surgical procedure in a human in need thereof comprising administering to the human as an intravenous infusion: (a) a therapeutically effective amount of lidocaine or a pharmaceutically acceptable salt thereof; and (b) a therapeutically effective amount of methylnaltrexone or a pharmaceutically acceptable salt thereof.

The invention also relates to synergistic combinations of lidocaine and methylnaltrexone in a unitary dosage form. Thus, in a sixth principal embodiment the invention provides a pharmaceutical composition comprising (a) a therapeutically effective amount of lidocaine or a pharmaceutically acceptable salt thereof; (b) a therapeutically effective amount of methylnaltrexone or a pharmaceutically acceptable salt thereof; and (c) one or more pharmaceutically acceptable carriers.

Additional advantages of the invention are set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying drawings, which are incorporated in and constitute a part of this specification, illustrate several embodiments of the invention and together with the description serve to explain the principles of the invention.

FIG. 1 depicts SDS page banding patterns of Src-protein activation resulting from 20 ng/ml mouse TNF-α in a KPC-105 mouse cell line (FIG. 1A) and 20 ng/ml human TNF-α in pancreatic cancer cells (FIG. 1B) as described in Example 1.

FIG. 2 depicts SDS page banding patterns of Src-protein activation resulting from varying concentrations of lidocaine in a human pancreatic cancer cell line after 30 minutes of incubation, as described in Example 2.

FIG. 3 depicts SDS page banding patterns of Src-protein activation resulting from varying concentrations of lidocaine in a KPC-105 mouse cell line after 30 minutes of incubation, as described in Example 3.

FIGS. 4A and 4B depict SDS page banding patterns of Src-protein activation resulting from 10 µM lidocaine treated KPC-105 cells at different time points, using 25 µg /lane (NP40 lysates) 10% SDS PAGE, as described in Example 4.

FIG. 5 depicts SDS page banding patterns of Src-protein activation resulting from 10 µM lidocaine treated KPC-105 cells (FIG. 5A) and methylnaltrexone treated cells (FIG. 5B), using 15 µg /lane (NP40 lysates) 10% SDS PAGE, as described in Example 5.

FIG. 6 depicts SDS page banding patterns of Src-protein activation resulting from 100 nM methylnaltrexone treated KPC-105 cells, using 10 µg /lane (NP40 lysates) 10% SDS PAGE, as described in Example 6.

FIG. 7 depicts SDS page banding patterns of Src-protein activation resulting from 10 µM lidocaine + 100 nM methylnaltrexone treated KPC-105 cells, using 10 µg /lane (NP40 lysates) 10% SDS PAGE, as described in Example 7.

FIG. 8 depicts SDS page banding patterns of Src-protein activation resulting from 10 µM lidocaine, 100 nM methylnaltrexone, and 10 µM lidocaine + 100 nM methylnaltrexone treated KPC-105 cells, using 7.5 µg /lane (RIPA lysates) 10% SDS PAGE, as described in Example 8.

FIG. 9 depicts SDS page banding patterns of Src-protein activation resulting from 10 µM lidocaine, 100 nM methylnaltrexone, and 10 µM lidocaine + 100 nM methylnaltrexone treated KPC-105 cells, using 7.5 µg /lane (RIPA lysates) 10% SDS PAGE, as described in Example 9.

FIG. 10 depicts SDS page banding patterns of Src-protein activation resulting from 10 µM lidocaine, 100 nM methylnaltrexone, and 10 µM lidocaine + 100 nM methylnaltrexone treated human pancreatic cancer cells after one hour, using 30 µg /lane (RIPA lysates) 10% SDS PAGE, as described in Example 10.

FIG. 11 depicts SDS page banding patterns of Src-protein activation resulting from 10 µM lidocaine, 100 nM methylnaltrexone, and 10 µM lidocaine + 100 nM methylnaltrexone treated human pancreatic cancer cells after one hour, using 30 µg /lane (RIPA lysates) 10% SDS PAGE, as described in Example 11.

FIG. 12 depicts SDS page banding patterns of Src-protein activation resulting from 10 µM lidocaine, 100 nM methylnaltrexone, and 10 µM lidocaine + 100 nM methylnaltrexone treated human pancreatic cancer cells after multiple time points, using 15 µg /lane (RIPA lysates) 10% SDS PAGE, as described in Example 12.

FIG. 13 depicts hematoxylin and eosin (H&E) staining and pathological scoring of lungs and spleen of unchallenged or LPS-challenged mice treated with lidocaine, methylnaltrexone, or a combination of lidocaine and methylnaltrexone, rated pathologically on a sliding scale of from 0 (no expression) to 4+ (strong uniform expression), as described in Example 13.

FIG. 14 depicts LPS-induced serum inflammatory cytokines profiles for (A) interleukin 1 alpha (IL-1α) (A), interferon-gamma (IFNγ) (B), tumor necrosis factor- alpha (TNF-α) (C), monocyte chemoattractant protein 1 (MCP-1) (D), interleukin 10 (IL- 10) (E), interleukin 6 (IL-6) (F), and interleukin 17A (IL-17A) (G), measured in serum of control and LPS-challenged mice treated with lidocaine, methylnaltrexone, or a combination of lidocaine and methylnaltrexone, using a LEGENDplex™ mouse inflammation panel (BioLegend, USA) kit followed by flow cytometry, as described in Example 13.

FIG. 15 depicts the status of macrophages in lungs (A) and spleen (B) following immunohistochemistry (IHC) staining using anti-mouse F4/80 antibody and scoring in lungs and spleen of unchallenged or LPS-challenged mice treated with lidocaine, methylnaltrexone, or a combination of lidocaine and methylnaltrexone, rated pathologically on a sliding scale of from 0 (no expression) to 4+ (strong uniform expression), as described in Example 13.

FIG. 16 depicts the status of natural killer (NK) cells in lungs (A) and spleen (B) following IHC staining using anti-mouse NK1.1 antibody and scoring in lungs and spleen of unchallenged or LPS-challenged mice treated with lidocaine, methylnaltrexone, or a combination of lidocaine and methylnaltrexone, rated pathologically on a sliding scale of from 0 (no expression) to 4+ (strong uniform expression), as described in Example 13.

FIG. 17 depicts the status of B cells in lungs (A) and spleen (B) following IHC staining using anti-mouse CD19 antibody and scoring in lungs and spleen of unchallenged or LPS-challenged mice treated with lidocaine, methylnaltrexone, or a combination of lidocaine and methylnaltrexone, rated pathologically on a sliding scale of from 0 (no expression) to 4+ (strong uniform expression), as described in Example 13.

FIG. 18 depicts the status of T cells in lungs (A) and spleen (B) following IHC staining using anti-mouse CD3 antibody and scoring in lungs and spleen of unchallenged or LPS-challenged mice treated with lidocaine, methylnaltrexone, or a combination of lidocaine and methylnaltrexone, rated pathologically on a sliding scale of from 0 (no expression) to 4+ (strong uniform expression), as described in Example 13.

FIG. 19 depicts the status of CD4+ T cells in lungs (A) and spleen (B) following IHC staining using anti-mouse CD4 antibody in lungs and spleen of unchallenged or LPS-challenged mice treated with lidocaine, methylnaltrexone, or a combination of lidocaine and methylnaltrexone, rated pathologically on a sliding scale of from 0 (no expression) to 4+ (strong uniform expression), as described in Example 13.

FIG. 20 depicts the status of CD8+ T cells in lungs (A) and spleen (B)following IHC staining using anti-mouse CD8 antibody in lungs and spleen of unchallenged or LPS-challenged mice treated either with lidocaine, methylnaltrexone, or a combination of lidocaine and methylnaltrexone, rated pathologically on a sliding scale of from 0 (no expression) to 4+ (strong uniform expression), as described in Example 13.

### DETAILED DESCRIPTION

### Definitions and Use of Terms

As used in this specification and in the claims which follow, the singular forms “a,” “an” and “the” include plural referents unless the context clearly dictates otherwise.

As used in this specification and in the claims which follow, the word “comprise” and variations of the word, such as “comprising” and “comprises,” means “including but not limited to,” and is not intended to exclude, for example, other additives, components, integers or steps. When an element is described as comprising a plurality components, steps or conditions, it will be understood that the element can also be described as comprising any combination of such plurality, or “consisting of” or “consisting essentially of” the plurality or combination of components, steps or conditions.

“Therapeutically effective amount” means that amount which, when administered to a human for supporting or affecting a metabolic process, or for treating or preventing a disease, is sufficient to cause such treatment or prevention of the disease, or supporting or affecting the metabolic process.

When ranges are given by specifying the lower end of a range separately from the upper end of the range, or specifying particular numerical values, it will be understood that a range can be defined by selectively combining any of the lower end variables, upper end variables, and particular numerical values that is mathematically possible. In like manner, when a range is defined as spanning from one endpoint to another, the range will be understood also to encompass a span between and excluding the two endpoints.

When “drug therapy” or a “method of treatment” is recited, it will be understood that the therapy can be accomplished through any suitable route of administration using any acceptable dosage form, and that the drug can be administered as the free base, a salt, or an ester or other prodrug moiety.

When used herein the term “about” will compensate for variability allowed for in the pharmaceutical industry and inherent in products in this industry, such as differences in product strength due to manufacturing variation and time-induced product degradation, salt selection, and molecular solvates and degrees of hydration.

In the context of the present invention insofar as it relates to any of the disease conditions recited herein, the term “treatment” means to reduce the occurrence of a symptom or condition, or to relieve or alleviate at least one symptom associated with such condition, or to slow or reverse the progression of such condition, or to manage or affect the metabolic processes underlying such condition. Within the meaning of the present invention, the terms also denote to arrest, delay the onset (i.e., the period prior to clinical manifestation of a disease) and/or reduce the risk of developing or worsening a disease.

The phrase “acceptable” as used in connection with compositions of the invention, refers to molecular entities and other ingredients of such compositions that are physiologically tolerable and do not typically produce untoward reactions when administered to a subject (e.g., a mammal such as a human).

When percentages are given herein, it will be understood that the percentages are weight percent, and that proportions are based on weight, unless otherwise stated to the contrary or evident from the surrounding context.

When a compound is expressed without indicating whether it is present as a free base or a salt, it will be understood to include both the free base and salt forms. In like manner, when a range of weights, doses, or ratios for a compound is given, it will be understood to include ranges calculated based on the weight of the free base and the salt, unless a particular salt is mentioned, in which case the range shall refer to the weight of the mentioned salt. Thus, when reference is made to 100 mg lidocaine, or 100 mg of lidocaine or a pharmaceutically acceptable salt thereof, the disclosure will be understood to encompass 100 mg of lidocaine as the free base, 100 mg of lidocaine hydrochloride based on the weight of the free base, or 100 mg lidocaine hydrochloride based on the weight of the salt, among other salts. When reference is made to 100 mg lidocaine hydrochloride, the disclosure will be understood only to encompass 100 mg of lidocaine hydrochloride based on the weight of the salt.

A preferred salt of methylnaltrexone in any of the embodiments of the invention is methylnaltrexone hydrobromide. A preferred salt of lidocaine in any of the embodiments of the invention is lidocaine hydrochloride.

Wherever an analysis or test is required to understand a given property or characteristic recited herein, it will be understood that the analysis or test is performed in accordance with applicable guidances, draft guidances, regulations and monographs of the United States Food and Drug Administration (“FDA”) and United States Pharmacopoeia (“USP”) applicable to drug products in the United States in force as of Jan. 1, 2020, unless otherwise specified.

### Principal Embodiments

The invention is described in terms of principal embodiments and subembodiments, and it will be understood that the principal embodiments can be combined to define other principal embodiments, that the subembodiments can be combined to define additional subembodiments, and that the subembodiments and combinations of subembodiments can be combined with all of the principal embodiments to define further embodiments of the present invention. The ability to combine embodiments and subembodiments is limited only by what is mathematically or physically impossible.

In a first principal embodiment the invention provides a method of treating inflammation resulting from an invasive surgical procedure in a human in need thereof comprising administering to the human as an intravenous infusion: (a) a therapeutically effective amount of lidocaine or a pharmaceutically acceptable salt thereof; and (b) a therapeutically effective amount of methylnaltrexone or a pharmaceutically acceptable salt thereof.

In a second principal embodiment the invention provides a method of inhibiting proliferation or metastasis of cancer cells following surgical intervention to remove a cancerous tumor in a human in need thereof comprising administering to the human as an intravenous infusion: (a) a therapeutically effective amount of lidocaine or a pharmaceutically acceptable salt thereof; and (b) a therapeutically effective amount of methylnaltrexone or a pharmaceutically acceptable salt thereof.

In a third principal embodiment the invention provides a method of inhibiting Src tyrosine protein kinase phosphorylation at Tyr419 following an invasive surgical procedure in a human in need thereof comprising administering to the human as an intravenous infusion: (a) a therapeutically effective amount of lidocaine or a pharmaceutically acceptable salt thereof; and (b) a therapeutically effective amount of methylnaltrexone or a pharmaceutically acceptable salt thereof.

In a fourth principal embodiment the invention provides a method of inhibiting cell signalling mediated by Src tyrosine protein kinase phosphorylation following an invasive surgical procedure in a human in need thereof comprising administering to the human as an intravenous infusion: (a) a therapeutically effective amount of lidocaine or a pharmaceutically acceptable salt thereof; and (b) a therapeutically effective amount of methylnaltrexone or a pharmaceutically acceptable salt thereof.

In a fifth principal embodiment the invention provides a method of treating a disease mediated by Src tyrosine protein kinase phosphorylation following an invasive surgical procedure in a human in need thereof comprising administering to the human as an intravenous infusion: (a) a therapeutically effective amount of lidocaine or a pharmaceutically acceptable salt thereof; and (b) a therapeutically effective amount of methylnaltrexone or a pharmaceutically acceptable salt thereof.

In a sixth principal embodiment the invention provides a pharmaceutical composition comprising (a) a therapeutically effective amount of lidocaine or a pharmaceutically acceptable salt thereof; (b) a therapeutically effective amount of methylnaltrexone or a pharmaceutically acceptable salt thereof; and (c) one or more pharmaceutically acceptable carriers.

### Discussion of Subembodiments

Various techniques are available for performing the methods of the present invention. For example, the invention can be practiced pre-operatively, during the surgery, and/or after the surgery, through a continuous intravenous infusion.

Thus, in various subembodiments the invention provides:
administering the composition as a continuous infusion before the surgery;
administering the composition as a continuous infusion during the surgery;
administering the composition as a continuous infusion after the surgery, preferably for a period of at least 24 or 48 hours;
any combination of the above.

Most preferably, the composition will be administered before the surgery, during the surgery, and after the surgery, defined herein as the “perioperative” period.

For purposes of this invention, unless qualified to exclude a slow bolus, a continuous intravenous infusion will be understood to allow for a slow bolus, although in a preferred embodiment the term will be used in its traditional sense.

It is also important to monitor for cardiac complications. Thus, in a preferred embodiment, the patient is preferably monitored telemetrically during any or all of these periods. It is most convenient to monitor telemetrically during the surgery but, where possible, telemetric monitoring should occur during all stages.

When the composition is administered by an infusion before the surgery, the infusion preferably occurs for a period lasting anywhere from 30 minutes to 12 hours or from 30 minutes to 6 hours. A pre-surgery infusion should occur as close to the surgery as possible and should preferably end no later than 2 hours or 30 minutes prior to the surgery.

When the composition is administered by infusion after the surgery, the infusion preferably occurs for at least 6 hours and can last up to 72 hours, but preferably lasts about 48 or 24 hours. A post-surgery infusion should occur as close to the surgery as possible and should preferably begin no later than 2 hours or even 30 minutes after to the surgery. In a preferred embodiment, the continuous infusion will continue unabated as the patient progresses through the pre-surgery, perioperative, and post-surgery periods.

The amount of lidocaine administered can be expressed on a daily basis. Thus, in general, the lidocaine dose will range on a daily basis from 10 to 3000 mg, from 100 to 2500 mg, or from 200 to 2000 mg. In alternative embodiments the amount ranges from: 10-100 mg, 10-50 mg, 50-100 mg, 100-200 mg, 100-150 mg, 150-200 mg, 200-300 mg, 200-250 mg, 250-300 mg, 300-400 mg, 300-350 mg, 350-400 mg, 400-500 mg, 400-450 mg, 450-500 mg, 500-600 mg, 500-550 mg, 550-600 mg, 600-700 mg, 600-650 mg, 650-700 mg, 700-800 mg, 700-750 mg, 750-800 mg, 800-900 mg, 800-850 mg, 850-900 mg, 900-100 mg, 900-950 mg, 950-1000 mg, 1000-1100 mg, 1100-1200 mg, 1200-1300 mg, 1300-1400 mg, 1400-1500 mg, 1500-1600 mg, 1600-1700 mg, 1700-1800 mg, 1800-2000 mg, 2000-2200 mg, 2200-2400 mg, 2400-2600 mg, 2500 or 2800 mg, or 2800-3000 mg, endpoints preferably included.

In a particularly preferred set of embodiments, the lidocaine dose on a daily basis is 850-3000 mg, 950-2500 mg, or 1000-2000 mg, endpoints preferably included.

The amount of lidocaine administered also can be expressed as a rate per body weight. Thus, the lidocaine is preferably administered as a continuous infusion at a rate of from 0.5-50 mg/kg/day, 1-40 mg/kg/day, or 5-30 mg/kg/day. Other alternatives include 0.5-2 mg/kg/day, 2-5 mg/kg/day, 5-10 mg/kg/day, 10-15 mg/kg/day, 15-20 mg/kg/day, 20-25 mg/kg/day, 25-30 mg/kg/day, 30-35 mg/kg/day, 35-40 mg/kg/day, 40-45 mg/kg/day.

Particularly preferred rates of administration are 10-45 mg/kg/day, 15-35 mg/kg/day, and 20-30 mg/kg/day. The dose will always be less than the amount that produces a serum concentration greater than 5 mg/L to prevent unwanted complications such as lightheadedness.

The amount of methylnaltrexone administered can also be expressed on a daily basis. Thus, in general, the dose of methylnaltrexone will range on a daily basis from 0.2 to 175 mg, from 0.5 mg to 100 mg, from 2 to 20 mg, or from 5 to 15 mg. In alternative embodiments the amount ranges from: 0.5-10 mg, 0.5-5 mg, 5-10 mg, 10-20 mg, 10-15 mg, 15-20 mg, 20-30 mg, 20-25 mg, 25-30 mg, 30-40 mg, 30-35 mg, 35-40 mg, 40-50 mg, 40-45 mg, 45-50 mg, 50-60 mg, 50-55 mg, 55-60 mg, 60-70 mg, 70-80 mg, 80-90 mg, 90-100 mg, or 100-175 mg, endpoints preferably included.

Particularly preferred rates of intravenous infusion are from 15 to 150 mg/day, from 20 to 120 mg/day, and from 25 to 100 mg/day, endpoints preferably included.

Expressed on a rate per body weight basis, the methylnaltrexone is preferably administered as a continuous intravenous infusion at a rate of from 0.02 to 2.5 mg/kg/day, from 0.05 to 1 mg/kg/day, from 0.1 to 0.5 mg/kg/day, or about 0.3 mg/kg/day. In alternative embodiments the amount ranges from 0.02-0.05 mg/kg/day, 0.05-0.1 mg/kg/day, 0.1-0.5 mg/kg/day, 0.5-1 mg/kg/day, 1-1.5 mg/kg/day, 1.5-2 mg/kg/day, or 2-2.5 mg/kg/day, endpoints preferably included.

Particularly preferred rates of intravenous infusion for the methylnaltrexone are 0.2-2 mg/kg/day, 0.25-1.75 mg/kg/day, and 0.30-1.5 mg/kg/day, endpoints preferably included. The methylnaltrexone plasma concentration will always be kept below 1400 ng/ml to prevent unwanted cardiovascular complications.

As with all rates of expression given herein for the lidocaine and methylnaltrexone, the foregoing rates of administration apply regardless of whether the composition is administered multiple days, an entire day or a fraction thereof. However, higher rates will typically be adopted when the drug is infused for periods less than an entire day, to accommodate the smaller amount of time needed to infuse an entire dose.

The ratio of methylnaltrexone to lidocaine in the compositions of the present invention, or administered according to the present invention, is preferably from 1:5 to 1:350 or from 1:20 to 1:200. In alternative embodiments, the weight ratio ranges from: 1:5-1:25, 1:5-1:15, 1:15-1:25, 1:25-1:45, 1:25-1:35, 1:35-1:45, 1:45-1:65, 1:45-1:55, 1:55-1:65, 1:65-1:85, 1:65-1:75, 1:75-1:85, 1:85-1:105, 1:85-1:95, 1:95-1:105, 1:05-1:25, 1:05-1:15, or 1:15-1:25, endpoints preferably included.

Particularly preferred weight ratios of lidocaine to methylnaltrexone range from: 1:10-1:125; 1:20-1:100; and 1:30-1:75.

Preferred total amounts of lidocaine hydrochloride and methylnaltrexone bromide for administration during the pre-surgery period, the actual surgical period, and/or the post-surgery period, and their ratios in any of the combined formulations, are:
0.5-100 mg methylnaltrexone bromide and 10-3000 mg of lidocaine hydrochloride at a ratio of 1:5 to 1:350.
0.5-100 mg methylnaltrexone bromide and 10-3000 mg of lidocaine hydrochloride at a ratio of 1:20 to 1:200.
2-20 mg methylnaltrexone bromide and 100-2500 mg of lidocaine hydrochloride at a ratio of 1:5 to 1:350.
2-20 mg methylnaltrexone bromide and 100-2500 mg of lidocaine hydrochloride at a ratio of 1:20 to 1:200.
0.02-2.5 mg/kg/day methylnaltrexone bromide and 0.5-50 mg/kg/day of lidocaine hydrochloride at a ratio of 1:5 to 1:350.
0.02-2.5 mg/kg/day methylnaltrexone bromide and 0.5-50 mg/kg/day of lidocaine hydrochloride at a ratio of 1:20 to 1:200.
0.1-0.5 mg/kg/day methylnaltrexone bromide and 5-30 mg/kg/day of lidocaine hydrochloride at a ratio of 1:5 to 1:350.
0.1-0.5 mg/kg/day methylnaltrexone bromide 5-30 mg/kg/day of lidocaine hydrochloride at a ratio of 1:20 to 1:200.
Once again, treatment during the post-surgery period preferably lasts for 24 or 48 hours, and administration during any of these periods is preferably accompanied by telemetric monitoring.

The invention is particularly useful in patients undergoing invasive surgical procedures. For purposes of this invention, an invasive surgical procedure refers to an operative procedure in which skin or mucous membranes and connective tissue are penetrated or incised, and include procedures to excise cancerous tissue, organ transplantation, hip and knee replacements, and the like. The invention includes both minor and major surgical interventions. Major surgery is generally any invasive operative procedure in which a more extensive resection is performed, e.g. a body cavity is entered, organs or tissue are removed, or normal anatomy is altered. In general, if a mesenchymal barrier is opened (pleural cavity, peritoneum, meninges), the surgery is considered major. Major surgeries are not typically performed via laparoscopy. As a consequence, the methods of the invention are particularly suitable for non-laparoscopic surgeries.

The invention has particular utility in tumor resection, particular in the resection of tumors of the pancreas, kidney, liver, lung, colorectal, breast, and bladder. Thus, for example, the methods of the present invention can be used to treat patients with exocrine pancreatic cancers including adenocarcinoma (ductal and acinar), intraductal papillary mucinous neoplasm acinar cell carcinoma, adenosquamous carcinoma, colloid carcinoma, giant cell tumor, hepatoid carcinoma, mucinous cystic neoplasms, pancreatoblastoma, serous cystadenoma, signet ring cell carcinoma, solid and pseudopapillary tumors, squamous cell carcinoma, and undifferentiated carcinoma. The methods also can be used to treat endocrine pancreatic cancers, including pancreatic neuroendocrine tumors (functioning or nonfunctioning) or islet cell tumors. Functioning neuroendocrine tumor include: Insulinoma, Glucagonoma, Gastrinoma, Somatostatinoma, VIPomas, and PPomas. The methods also can be used to treat a kidney tumor, such as chromophobe renal cell carcinoma, clear cell renal cell carcinoma, nephroblastoma (Wilms tumor); papillary renal cell carcinoma, primary renal ASPSCR1-TFE3 tumor, or renal cell carcinoma. Alternatively, the methods can be used to treat a liver tumor such as hepatoblastoma or hepatocellular carcinoma. In still further embodiment, the methods can be used to treat a lung tumor such as non-small cell carcinoma or small cell cancer.

Colorectal cancers treatable according to the current invention include adenocarcinomas of the colon and rectum, which make up 95 percent of all colorectal cancer cases, but also include primary colorectal lymphomas, gastrointestinal stromal tumors, leiomyosarcomas, carcinoid tumors and melanomas. Breast cancers treatable by the current invention include invasive breast cancers, noninvasive breast cancers, ductal carcinoma in situ (DCIS), invasive ductal carcinoma, invasive lobular carcinoma, lobular carcinoma in situ, atypical lobular hyperplasia, inflammatory breast cancer, breast sarcoma, metaplastic carcinoma, estrogen receptor-positive breast cancer, triple-negative breast cancer, and breast papilloma. Bladder cancers treatable by the current invention include urothelial carcinoma, squamous cell carcinoma, adenocarcinoma, and small cell carcinoma. Particularly preferred cancerous tumors for treatment by the current invention, regardless of the cancer type, are cancerous tumors that rely on angiogenic processes or Src signaling. The size of the tumor removed in the surgical procedure can vary but, in various embodiments, greater than 5 g, 20 g, 50 g, or even 100 g of tissue is removed.

The patient might also be on chemotherapy. Thus, in one embodiment the patient has received or is currently receiving an anticancer agent. In another preferred embodiment the method is performed in the absence of concurrent opioids.

The compositions are preferably present in form of a sterile liquid or powder for injectable administration upon reconstitution. The compositions are preferably administered as an injectable intravenous infusion which can, as mentioned previously, include a slow bolus. The composition is preferably in the form of a unit dose or multi-dose sterile liquid or powder for injectable administration.

Preparations for injectable administration include sterile aqueous or non-aqueous solutions, suspensions, and emulsions. While solvents are most likely not needed for formulating lidocaine and methylnaltrexone, examples of suitable non-aqueous solvents when solvents are used include propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of injectable vehicles include sodium chloride solution, Ringer’s dextrose, dextrose and sodium chloride, lactated Ringer’s, and fixed oils. Intravenous vehicles include fluid and nutrient replenishers, electrolyte replenishers (such as those based on Ringer’s dextrose), and the like. Preservatives and other additives such as, other antimicrobial, anti-oxidants, cheating agents, inert gases and the like also can be included or omitted.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

It is especially advantageous to formulate injectable compositions in unit dosage form for ease of administration and uniformity of dosage. “Unit dosage form” as used herein, refers to physically discrete units suited as unitary dosages for the individual to be treated; each unit containing a predetermined quantity of pharmaceutical composition is calculated to produce the desired therapeutic effect in association with the required pharmaceutical carrier. The specification for the dosage unit forms of the disclosure are related to the characteristics of the pharmaceutical composition and the particular therapeutic effect to be achieved.

Finally, while the invention has been expressed in terms of a single composition that contains the methylnaltrexone and lidocaine, it will be understood that the two can be administered separately with the same therapeutic effect.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention.

When referenced in the Examples, GeneTex refers to GeneTex Biotechnology company in Irvine California. Cell Signaling Technology refers to Cell Signaling Technology, Inc. in Danvers Massachusetts. Invitrogen refers to a line of brand products sold by Thermo Fisher Scientific corporation, headquartered in Carlsbad, California.

### Example 1

Example 1 evaluated the activation of p-Src in TNF-α treated KPC-105 mouse and human pancreatic cancer cell lines at varying time points.

### Experimental Conditions

On day 1, KPC-105 mouse and human pancreatic cancer cell lines were cultured until 300,000 cells per well in a 6 well plate were obtained. On day 2, each cell line was treated with 20 ng/ml or mouse or human TNF-α for 2-hours. Cells were then collected and washed with phosphate buffered saline and stored at -80° C. Cells were then lysed with 100 µl of radioimmunoprecipitation assay buffer with protease and phosphatase buffers.

Protein estimation was then performed using a Bradford assay under the following conditions:
A Western Blot was stripped with 6 M guanidine hydrochloride after p-Src protein blocking to probe with total Src protein.
10% SDS-PAGE with 15 well Blocking: 5% bovine serum albumin for p-Src blot P-Src-Tyr416 (GeneTex GTX81151) Rb: 1:1000 in 5% bovine serum albumin, Overnight.
Total Src (Cell Signaling Technology 2108S ) Rb mAb 1:1000 overnight in 5 % milk.
GAPDH (Invitrogen# AM4300) : 1:5000 in 5% milk overnight.
Wash: 4x for 5 minutes each with 1x TBST.
Developed using 50% femto for the p-Src protein and homemade ECL for total Src and GAPDH protein.

### Results

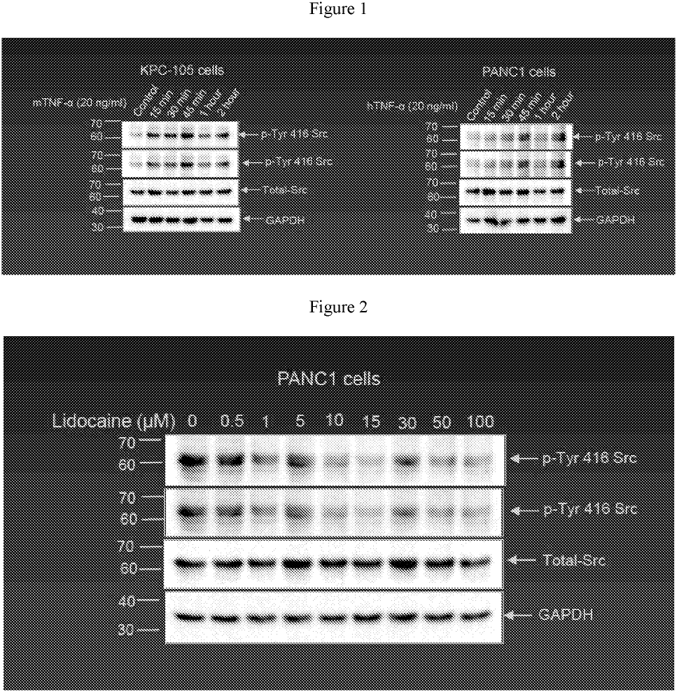

As reported in FIG. 1, there is a maximum activation of Src protein during treatment with TNF-α (20 ng/ml) after 45 minutes of exposure in both the KPC-105 mouse cell line and the human pancreatic cancer cell line.

### Example 2

Example 2 evaluated the ability of increasing doses of lidocaine to inhibit p-Src in human pancreatic cancer cells incubated for 30 minutes

### Experimental Conditions

On day 1, pancreatic cancer cells were cultured until 300,000 cells per well in a 6 well plate were obtained. On day 2, the cells were treated with 0, 0.5, 1,5, 10, 15, 30, 50 and 100 µM lidocaine for 30 minutes. Plates were then collected and washed with phosphate buffered saline and stored at -80° C. Cells were subsequently lysed with 100 µl of radioimmunoprecipitation assay buffer with protease and phosphatase buffers.

Protein estimation was done using a Bradford assay under the following conditions:
A Western Blot was stripped with 6 M guanidine hydrochloride after phosphor-Src protein blocking to probe with total Src protein.
10% SDS-PAGE 10 well Blocking: 5% bovine serum albumin for p-Src blot.
P-Src-Tyr416 (GeneTex GTX81151) Rb: 1:1000 in 5% bovine serum albumin, Overnight.
Total Src (Cell Signaling Technology 2108S ) Rb mAb 1:1000 overnight in 5% milk.
GAPDH (Invitrogen# AM4300) : 1:5000 in 5% milk overnight.
Wash: 4x for 5 minutes each with 1x TBST.
Developed using 50% femto for the p-Src protein and homemade ECL substrate for total Src and GAPDH protein.

### Results

As reported in FIG. 2, lidocaine reduced the level of p-Src protein after 30 minutes of treatment in human pancreatic cancer cells beginning at doses of 10 µM and 15 µM.

### Example 3

Example 3 evaluated the ability of increasing lidocaine doses to inhibit p-Src in mouse KPC-105 cells incubated for 30 minutes.

### Experimental Conditions

On day 1, mouse KPC-105 cells were cultured until 300,000 cells per well in a 6 well plate were obtained. On day 2, the cells were treated with 0, 0.5, 1,5, 10, 15, 30, 50 and 100 µM lidocaine for 30 minutes. Plates were then collected and washed with phosphate buffered saline and stored at -80° C. Cells were subsequently lysed with 100 µl of radioimmunoprecipitation assay buffer with protease and phosphatase buffers.

Protein estimation was done using a Bradford assay under the following conditions:
A Western Blot was stripped with 6 M guanidine hydrochloride after p-Src protein blocking to probe with total Src protein.
10% SDS-PAGE 10 well Blocking: 5% bovine serum albumin for p-Src blot.
P-Src-Tyr416 (GeneTex GTX81151) Rb: 1:1000 in 5% bovine serum albumin, Overnight.
Total Src (Cell Signaling Technology 2108S ) Rb mAb 1:1000 overnight in 5% milk.
GAPDH (Invitrogen# AM4300) : 1:5000 in 5% milk overnight.
Wash: 4x for 5 minutes each with 1x TBST.
Developed using 50% femto for the p-Src protein and homemade ECL substrate for total Src and GAPDH protein.

### Results

As reported in FIG. 3, lidocaine reduced the level of p-Src protein in KPC105 cells after 30 minutes of treatment beginning at a dose of 10 µM.

### Example 4

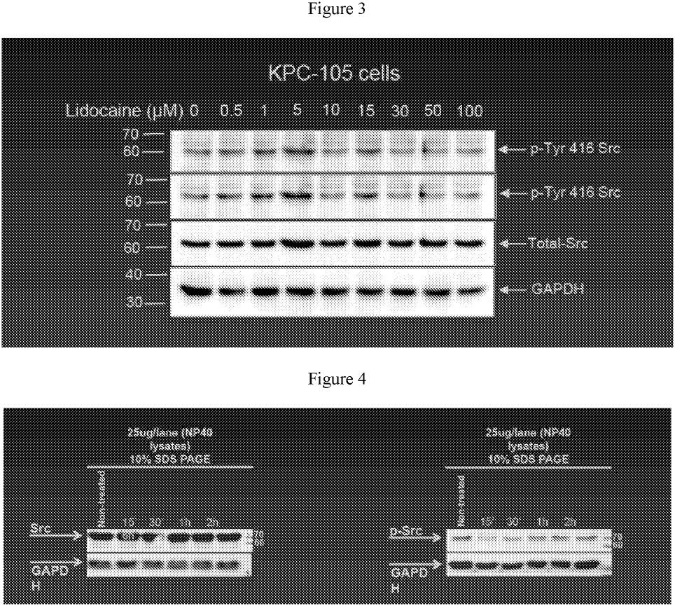

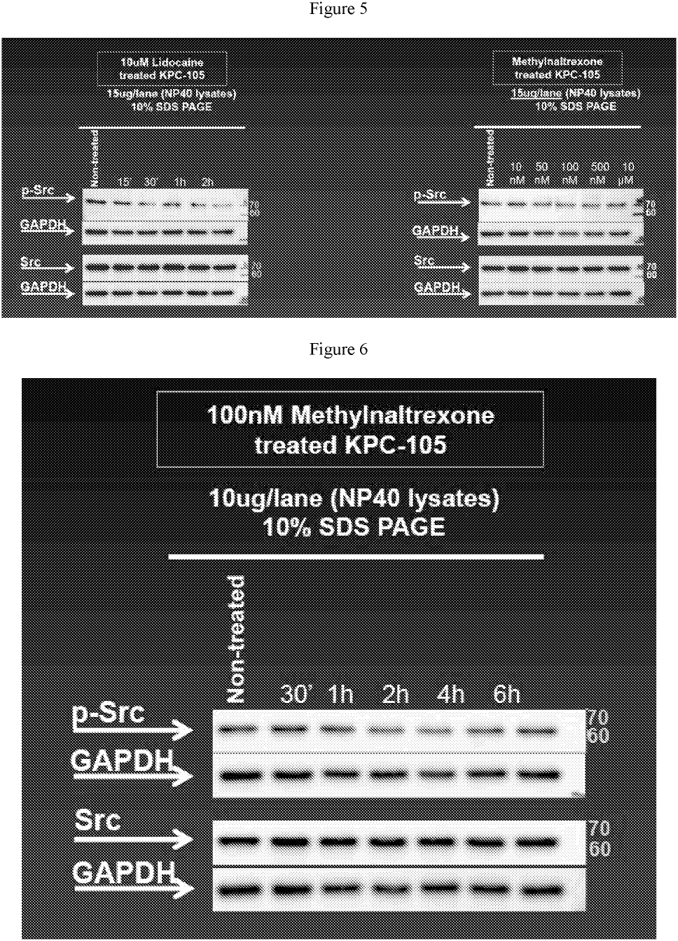

Example 4 evaluated endogenous Src and p-Src in 10 µM lidocaine treated mouse KPC-105 cells at different time points. As shown in FIGS. 4A and 4B, total Src was not affected by lidocaine exposure at any time point. With respect to p-Src, lidocaine attenuated Src phosphorylation after 15 minutes and up to 6 hours with maximum effects observed at 15 and 30 minutes.

### Example 5

Example 5 evaluated endogenous Src and p-Src in mouse KPC-105 cells treated with 10 10 µM lidocaine for different time points, and increasing doses of methylnaltrexone after one hour of incubation. Western Blot banding patterns were generated using 15 µg /lane (NP40 lysates) 10% SDS PAGE over a 6-hour duration (lidocaine) and a 1-hour duration (methylnaltrexone). As reported in FIGS. 5A and 5B, total Src was not affected by lidocaine or methylnaltrexone. In contrast, lidocaine attenuated Src phosphorylation at 30 minutes, 1 hour, 2 hours, and 6 hours, with inconsistent banding pattern at each time point. Methylnaltrexone attenuated Src phosphorylation at concentrations above 50 nM after 1 hour incubation.

### Example 6

After our previous experiment with methylnaltrexone in Example 5, we decided to load less protein and incubate cells with methylnaltrexone for more than 1 hour, and evaluate endogenous Src and p-Src in KPC-105 cells treated with 100 nM methylnaltrexone at different time points.

### Experimental Conditions

P-Src-Tyr416 (GeneTex # GTX81151) Rb: 1:1000 in 5% bovine serum albumin o/n at 4° C.
10 µg /lane (NP40 lysates) 10% SDS PAGE
Total Src (CST # 2108S ) Rb mAb 1:1000 in 5% bovine serum albumin o/n at 4° C.

### Results

As reported in FIG. 6, 100 nM methylnaltrexone attenuates phosphorylation of Src in KPC-105 cells starting at 2 hours with maximum effects observed at 2, 4 and 6 hours.

### Example 7

Example 7 evaluated endogenous Src and phospho-SRC in KPC-105 cells treated with the combination of 10 µM lidocaine + 100 nM methylnaltrexone at different time points.

### Experimental Conditions

P-Src-Tyr416 (GeneTex # GTX81151) Rb: 1:1000 in 5% bovine serum albumin 3 hours at room temperature
10 µg /lane (NP40 lysates) 10% SDS PAGE
Total Src (CST # 2108S ) Rb mAb 1:1000 in 5% bovine serum albumin 3 hours at room temperature

### Results

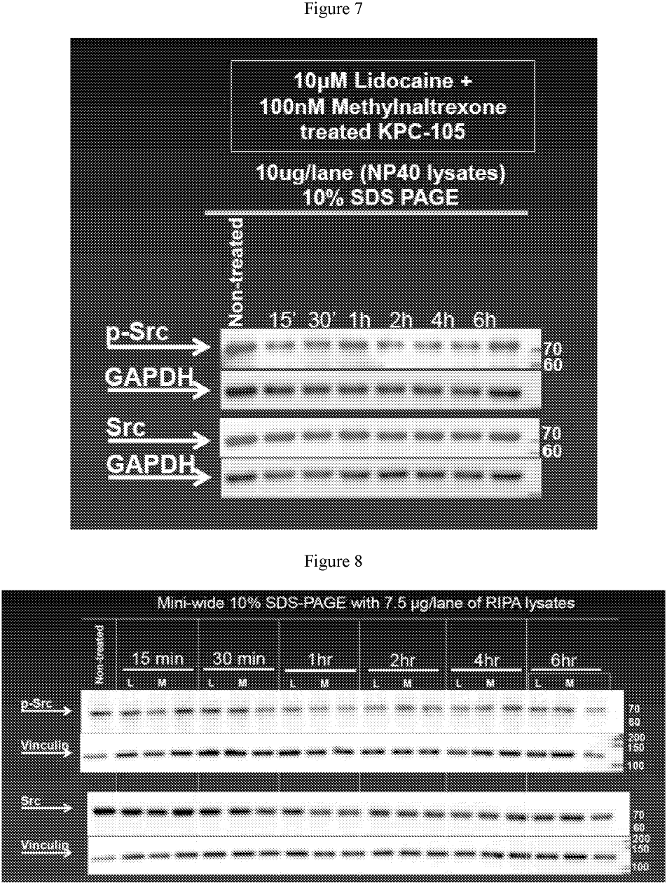

FIG. 7 reports that the combination of 10 µM lidocaine + 100 nM methylnaltrexone consistently attenuates phosphorylation of Src in KPC-105 cells beginning at 15 minutes and extending through 6 hours.

### Example 8

Example 8 evaluated endogenous Src and phospho-Src in KPC-105 cells treated individually with 10 µM lidocaine (L), 100 nM methylnaltrexone (M), or the combination of 10 µM lidocaine + 100 nM methylnaltrexone (L+M) at different time points.

### Experimental Conditions

P-Src-Tyr416 (GeneTex # GTX81151) 1:2000 in 5% bovine serum albumin, o/n at 4° C.
Total Src (CST # 2108S ) 1:3000 in 5% bovine serum albumin, o/n at 4° C.
Vinculin (ProteinTech) 1:4000 in 5% milk o/n at 4° C.

### Results

As shown in FIG. 8, lidocaine decreased total Src after 1 hours of exposure and attenuated phosphorylation of Src after 1 hour of exposure. Methylnaltrexone decreased total Src after 1 hour of exposure and attenuated phosphorylation of Src after 1 hour of exposure. Lidocaine + methylnaltrexone decreased total Src after 30 minutes of exposure (sooner than both individually) and attenuated phosphorylation of Src at 30 minutes, 1 hour, 2 hours and 6 hours. At 6 hours the combination of lidocaine and methylnaltrexone was remarkably effective compared to lidocaine individually, methylnaltrexone individually, or the non-treated control.

### Example 9

Example 9 evaluated endogenous Src and p-Src in KPC-105 cells treated individually with 10 µM lidocaine (L), 100 nM methylnaltrexone (M), and the combination of 10 µM lidocaine + 100 nM methylnaltrexone (L+M) at different time points, using a different loading protein than Example 8.

### Experimental Conditions

P-Src-Tyr416 (GeneTex # GTX81151) 1:2000 in 5% bovine serum albumin, o/n at 4C
Total Src (CST # 2108S ) 1:3000 in 5% bovine serum albumin, o/n at 4C

### Results

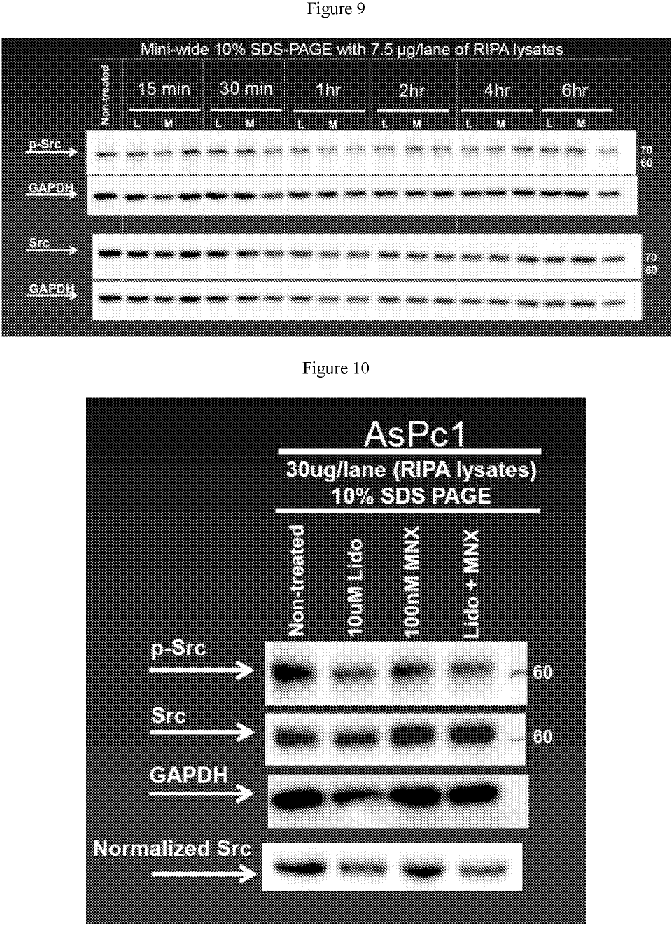

As reported in FIG. 9, practically the same results were obtained as in Example 8.

### Example 10

Example 10 evaluated the effect of 10 µM lidocaine (L), 100 nM methylnaltrexone (M) and the combination of 10 µM lidocaine + 100 nM methylnaltrexone (L+M) for 1 hour on total Src and p-Src expression in a human pancreatic cancer cell line (AsPc 1).

### Experimental Conditions

P-Src-Tyr416 (GeneTex # GTX81151) 1:2000 in 5% bovine serum albumin, at room temperature, 4 hours
Total Src (CST # 2108S) 1:3000 in 5% bovine serum albumin, at room temperature, 4 hours

### Results

As shown in FIG. 10, lidocaine and methylnaltrexone individually and in combination attenuated p-Src activity in AsPc 1 human pancreatic cancer cells after one hour. Preliminary results after Src normalization show no increase in Src with methylnaltrexone exposure.

### Example 11

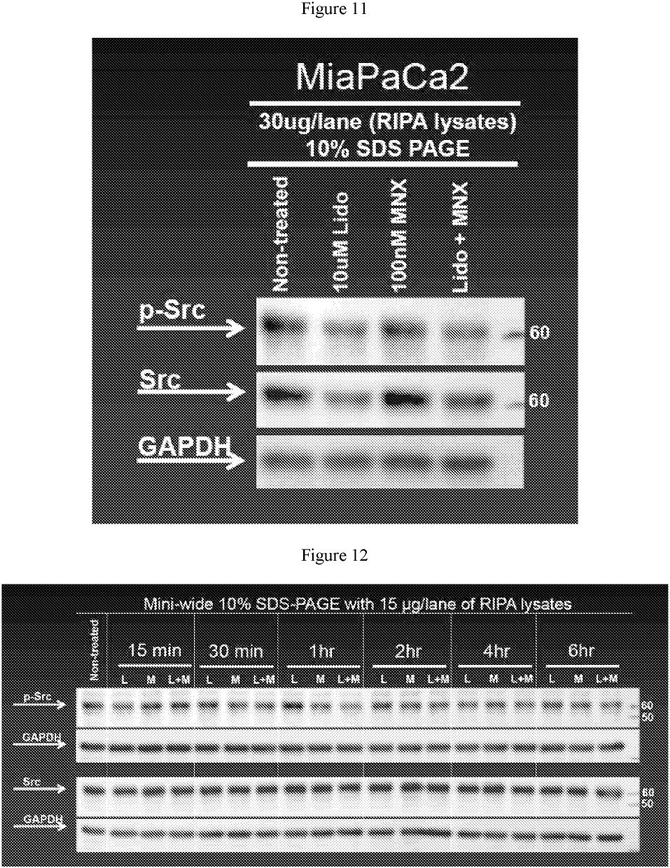

Example 11 evaluated the effect of 10 µM lidocaine (L), 100 nM methylnaltrexone (M) and the combination of 10 µM lidocaine + 100 nM methylnaltrexone (L+M) for 1 hour on total Src and p-Src expression in a human pancreatic cancer cell line (MiaPaCa 2). Experimental conditions were identical to Example 10. As reported in FIG. 11, both drugs individually and in combination attenuated p-Src in MiaPaCa 2 human pancreatic cells after one hour.

### Example 12

Example 12 evaluated the effect of 10 µM lidocaine (L), 100 nM methylnaltrexone (M) and the combination of 10 µM lidocaine + 100 nM methylnaltrexone (L+M) on total Src and p-Src expression in a human pancreatic cancer cell line (Panc1) at multiple time points up to 6 hours, in fresh media. Experimental conditions were identical to Example 10. As reported in FIG. 12, lidocaine by itself showed inconsistent effects on decreases in p-Src. Methylnaltrexone by itself initially decreased p-Src at 30 minutes and 1 hour. In contrast, the combination of lidocaine + methylnaltrexone consistently decreased p-Src from 30 minutes onwards.

### Example 13

The lipopolysaccharide (LPS) model of systemic inflammation has been reported as one of the most acceptable models to explore the impact of new therapies for acute inflammation. The LPS is a ubiquitous endotoxin from gram-negative bacteria and is known to induce pro-inflammatory diseases in humans and animals. We investigated the role of lidocaine or methylnaltrexone alone or a combination of lidocaine and methylnaltrexone in the LPS-induced inflammation model in immunocompetent, C57BL/6 mice.

### Experimental Details

C57BL/6J mice (6-8 weeks) were purchased from Charles River Laboratories (USA) and acclimatized for at least 1 week before use. All mice were housed in a pathogen-free facility. The mice received LPS for 24 hours. Following 24 hours, mice were treated either with lidocaine alone or methylnaltrexone alone or a combination of both, as shown in Table 1.

TABLE 1

In vivo treatment plan

| | | | |
|---|---|---|---|
| Group | LPS | Lidocaine | Methylnaltrexone |
| Group 1 | - | - | - |
| Group 2 | - | + | - |
| Group 3 | - | - | + |
| Group 4 | - | + | + |
| Group 5 | + | - | - |
| Group 6 | + | + | - |
| Group 7 | + | - | + |
| Group 8 | + | + | + |

At the conclusion of the study, the blood and tissue samples were collected for further study. The serum was used to determine pro-inflammatory cytokines using the LEGENDplex™ mouse inflammation panel (BioLegend, USA) kit followed by flow cytometry. The lungs and spleen tissue samples were used for hematoxylin and eosin (H&E) staining and immunohistochemical analyses for immune cells, including macrophages and natural killer (NK) cells, B cells, T cells, and its subsets. For histopathology and immunohistochemistry (IHC), tissue samples were prepared by cutting 4-µm sections from the paraffin blocks. IHC staining was performed by methods described earlier. The images were captured using bright field microscopy (Nikon Microscope). Two independent investigators evaluated the H&E and all immunohistochemical staining. For slide scoring, each investigator assessed the tissues and gave a score of 0 (no expression) to 4+ (strong uniform expression) as described previously. The data were expressed either as the mean ± SD or mean ± SEM by using Graph Pad Prism software.

### Results and Discussion

1. Combined treatment of lidocaine and methylnaltrexone decreases LPS-induced pathological abnormalities in lungs and spleen.

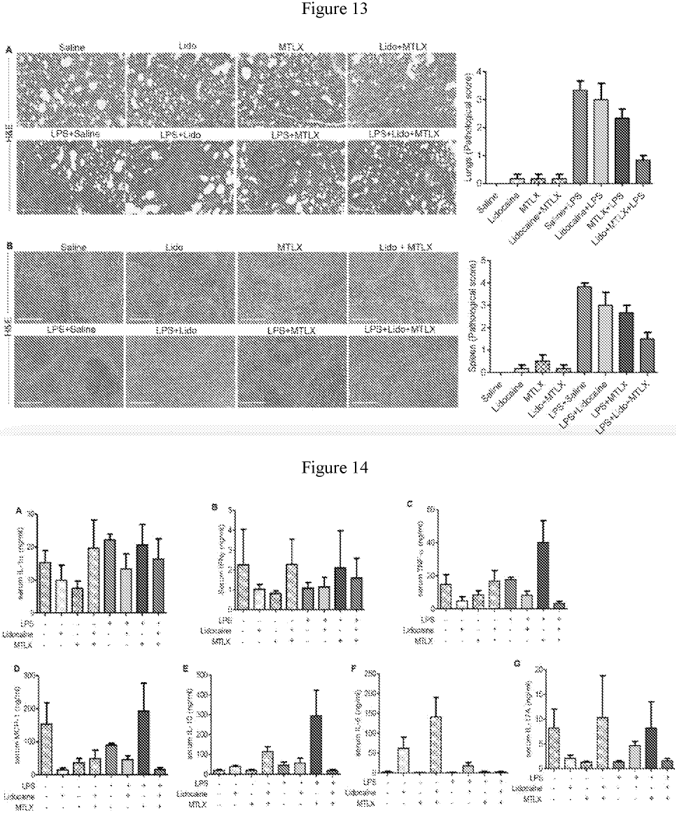

Acute lung injury is a critical illness that could lead to mortality (40-60%). Following injury to the alveolar epithelium and lung edema, neutrophil infiltration is reported as the main pathological changes due to lung inflammation. Therefore, to determine the therapeutic efficacy of lidocaine alone, methylnaltrexone alone, or a combination of lidocaine and methylnaltrexone in inflammation, C57BL/6 mice were challenged with LPS followed by treatment with drug alone or in combination as described in Table 1. At the conclusion of the study, mice were sacrificed, and tissue sections from the lungs and spleen were used for histopathological examinations. H&E staining showed perivascular edema and accumulation of mixed cell infiltration within blood, and lymphatic vessels in LPS challenged saline or lidocaine alone or methylnaltrexone alone treated groups (FIG. 13A). However, H&E staining showed modest histopathologic changes in the lungs of LPS challenged mice treated with lidocaine and methylnaltrexone together (FIG. 13A).

Lung inflammation is tightly regulated by immune infiltration, and organs with higher immune filtrates represent significantly greater inflammation. The spleen functions to clear senescent erythrocytes, maintain a blood reserve, and play a significant role in the immune system. Therefore, we investigated the therapeutic efficacy of lidocaine or methylnaltrexone alone or a combination of both drugs in the pathophysiology of the spleen using the LPS-induced inflammation mice model as shown in Table 1. Histological examination revealed an increased number of erythrocytes in the red pulp, along with mild edema in LPS challenged mice treated with saline or lidocaine alone or methylnaltrexone alone, however the mice treated with a combination of lidocaine and methylnaltrexone showed modest pathological changes in the spleen (FIG. 13B). Taken together, combined treatment of lidocaine and methylnaltrexone decreases LPS-induced pathological aberrations in the lungs and spleen.

2. Combined treatment of lidocaine and methylnaltrexone decreases LPS-induced pro-inflammatory serum cytokines.

Gram-negative bacterial infections are the main cause of acute lung injury, and LPS, which is the main component of the Gram-negative bacteria cell wall, is the major stimulus for the release of inflammatory mediators. Therefore, we measured the effect of lidocaine alone, methylnaltrexone alone, or a combination of lidocaine and methylnaltrexone on LPS-induced serum inflammatory cytokines profiles. Mouse inflammatory cytokines were measured in serum of control and LPS-challenged mice treated with drugs as described in Table 1, using the LEGENDplex™ mouse inflammation panel (BioLegend, USA) kit followed by flow cytometry as per manufacturer’s specifications. There were insignificant changes in the levels of interleukin 1 alpha (IL-1α) and interferon-gamma (IFNγ) in mice treated with lidocaine alone, methylnaltrexone alone, or a combination of lidocaine and methylnaltrexone (FIGS. 14A and 14B). However, serum tumor necrosis factor-alpha (TNF-α), monocyte chemoattractant protein 1 (MCP- 1), interleukin 10 (IL-10), interleukin 6 (IL-6), and interleukin 17A (IL-17A) levels were found decreased in mice challenged with LPS and treated with lidocaine and methylnaltrexone together (FIGS. 14C-G). IL-1, IL-6, IL-17A, MCP-1, and TNFα are pro- inflammatory cytokines associated with inflammatory signaling. Taken together, these findings indicate that combined treatment of lidocaine and methylnaltrexone has the potential to suppress inflammatory signaling.

3. Combined treatment of lidocaine and methylnaltrexone decreases LPS-induced macrophages and natural killer (NK) cells in lungs and spleen.

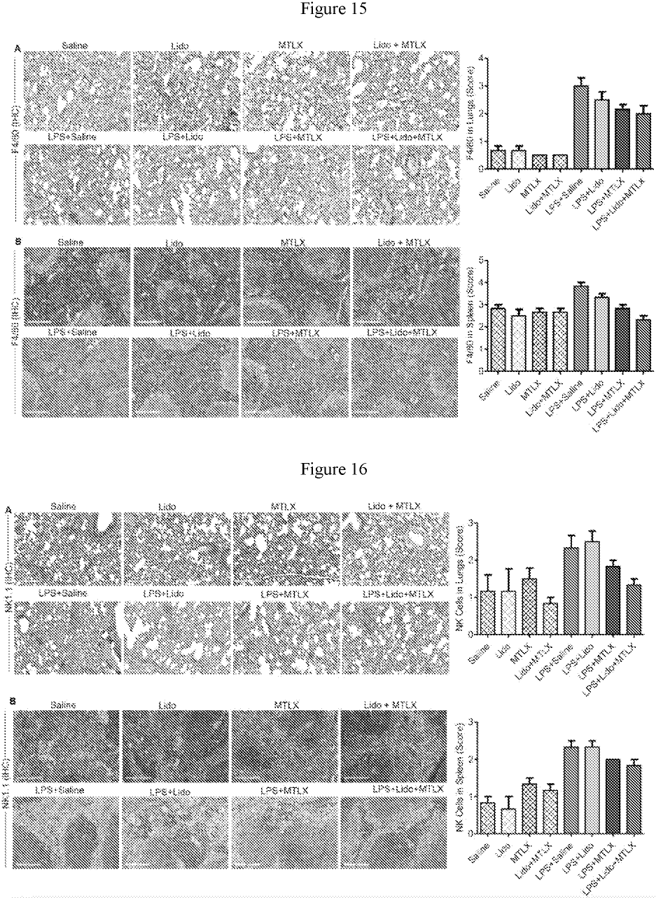

Both the innate and adaptive immune systems play an important role in inflammation. Among various members of the innate immune system, macrophages are crucial in regulating inflammation. It has been reported that LPS exerts adjuvant effects on macrophages, resulting in an inflammatory cascade defined by early production of pro- inflammatory cytokines, such as TNF-α and IL-6. Furthermore, LPS is known to stimulate monocytes/macrophages through toll-like receptor 4 (TLR4), resulting in the activation of a series of signaling events that potentiate production of inflammatory mediators. Because LPS-induced serum TNF-α and IL-6 levels were downregulated in mice treated with lidocaine and methylnaltrexone together, we determined macrophage status in lungs and spleen sections by IHC staining using the anti-mouse F4/80 antibody. The IHC results showed a partially decreased F4/80 positive area in lungs and spleen sections from LPS challenged mice treated either with lidocaine or methylnaltrexone alone (FIGS. 15A and 15B). However, combined treatment of lidocaine or methylnaltrexone in LPS challenged mice suppressed F4/80 positive area in lungs and spleen (FIGS. 15A and 15B). The NK cells are unique mediators of innate immunity, involved in cytotoxic activity and secretion of pro-inflammatory cytokines. To thoroughly dissect the influence of different lymphocyte populations on the LPS-induced host response, we determined NK cells’ infiltration in the lungs and spleen of lidocaine alone methylnaltrexone alone or a combination of both agents/drugs. We determined NK cell status in lungs and spleen sections by IHC staining using anti-mouse NK1.1 antibody. Results showed decreased NK1.1 positive area in lungs and spleen of LPS challenged mice treated with a combination of lidocaine and methylnaltrexone (FIGS. 16A and 16B). These IHC results collectively suggest that combined treatment of lidocaine and methylnaltrexone could impact macrophages and NK cell-mediated inflammatory signaling.

4. Combined treatment of lidocaine and methylnaltrexone decreases LPS-induced B cells in lungs and spleen.

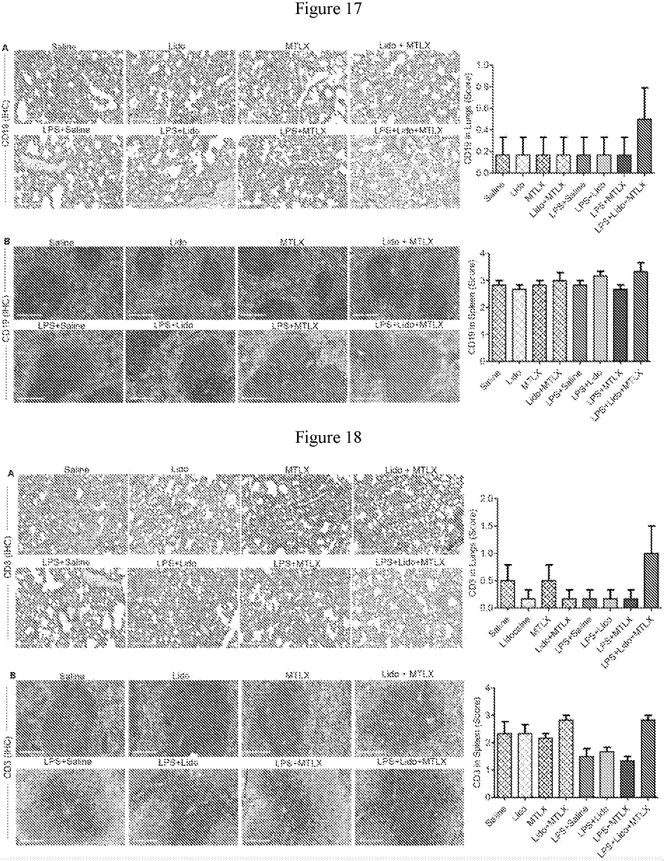

The TLRs play a crucial role in immune responses to pathogens by transducing signals in innate immune cells in response to microbial products, including LPS. Apart from their expression on macrophages, TLRs are also expressed on B cells that contribute to antibody-mediated immune responses. Therefore, to understand the effect of lidocaine alone or methylnaltrexone alone, or a combination of lidocaine and methylnaltrexone on B cells in the lungs and spleen, we performed IHC staining using an anti-mouse CD19 antibody. The IHC results showed a partially increased CD19 positive area in lungs and spleen sections from LPS-challenged mice treated together with lidocaine and methylnaltrexone (FIGS. 17A and 17B). Together, these results suggest that combined treatment of lidocaine and methylnaltrexone increases B cell population in LPS- induced inflammation.

5. Combined treatment of lidocaine and methylnaltrexone increases LPS-induced T cell and subsets in lungs and spleen.

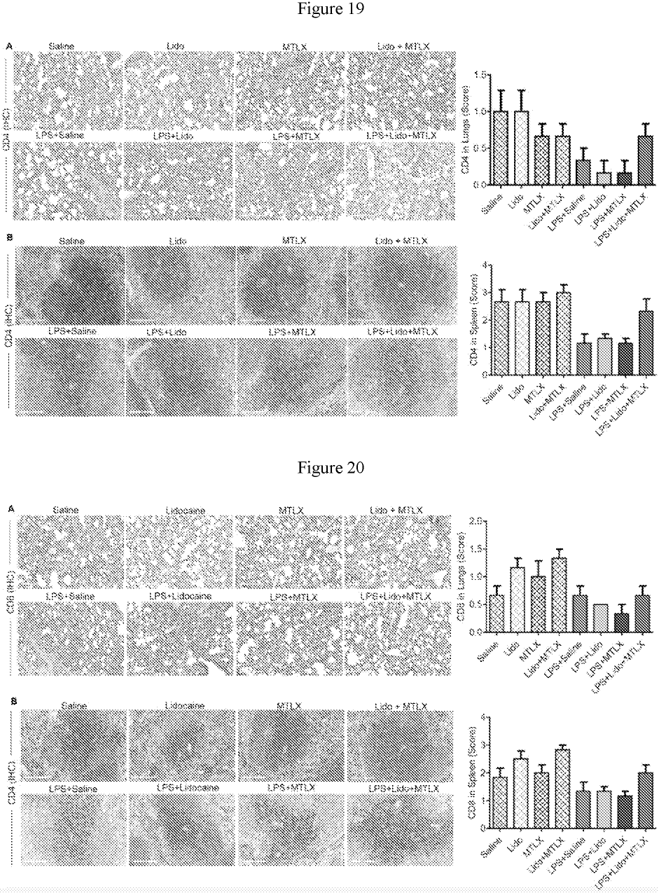

Like B cells, T cells are another member of the adaptive immune system. As inflammatory processes progress, pro-inflammatory cytokine production induces hypo-responsiveness in T-cells and subsets. To understand the impact of lidocaine alone or methylnaltrexone alone or a combination of both drugs in the infiltration of T cells, CD4+ and CD8+ T cells in lungs and spleen of LPS-challenged mice, we performed IHC staining in lungs and spleen sections using an anti-mouse CD3 antibody, anti-mouse CD4 antibody and anti-mouse CD8 antibody, respectively. The IHC results showed increased CD3, CD4 and CD8 positive area in lungs and spleen sections from mice treated with a combination of lidocaine and methylnaltrexone (FIGS. 18-20). The T cell suppression contributes to immune dysfunction. It has been reported that LPS can rapidly and dose-dependently suppress interleukin-2 (IL-2) production and T cell proliferation in peripheral blood mononuclear cells (PBMCs). Taken together, these results suggest that combined treatment of lidocaine and methylnaltrexone might have potential to improve T cell functions in LPS induced inflammation.

### Conclusion

In summary, the results indicate that lidocaine or methylnaltrexone alone could partially mitigate LPS-induced inflammation in a mouse model, and that the combined treatment of lidocaine and methylnaltrexone could potentially be used in the treatment of inflammatory states.

### Example 14

This example sets forth a protocol for preventing and managing inflammation and pain that arises from highly invasive surgical procedures (i.e. post-operative analgesia). This protocol includes cancer surgeries, although a separate protocol specifically for cancer is given in Example 15. The protocol is carried out at the rates of intravenous infusion described in Table 2, in one of the 9 potential combinations of dosing ranges, in the weight ratios of lidocaine to methylnaltrexone described in Table 3, for a total 27 combinations of dosing ranges and ratios. The dose of lidocaine and methylnaltrexone administered will always be below the maximum tolerated dose of each individual ingredient based on the risk to the patient’s cardiovascular system, particular the risk to cause cardiac arrhythmias and, for methylnaltrexone, the dose that either induces diarrhea or that treats opioid-induced constipation. The methylnaltrexone plasma concentration will always be kept below 1400 ng/ml to prevent unwanted cardiovascular complications. In like manner, the lidocaine plasma concentration will always be kept below 5 mg/L to avoid complications such as lightheadedness.

TABLE 2

Daily Infusion Rates
Rate of Administration (mg/kg/day)

| | Option 1 | Option 2 | Option 3 |
|---|---|---|---|
| Lidocaine HC1 | 10-45 | 15-35 | 20-30 |
| | Option 1 | Option 2 | Option 3 |
| Methylnaltrexone Br | 0.2-2 | 0.25-1.75 | 0.30-1.5 |

*Rates are based on the weight of the entire salt

TABLE 3

Ratios of Lidocaine to Methylnaltrexone

| | Option A | Option B | Option C |
|---|---|---|---|
| Weight ratio methylnaltrexone:lidocaine | 1:10-1:125 | 1:20-1:100 | 1:30-1:75 |

*Ratios are based on the weight of the entire salt

Surgical Procedure (non-laparoscopic):
thoracic, orthopedic, and abdominal surgeries
hemorrhoidectomies and bunionectomies
hip or knee arthroplasty, inguinal hernia repair
tumor resection, particularly tumors in the breast and pancreas
osteosarcoma (limb sparing surgery, amputation, or rotationplasty) Clinical Improvements:
Pain reduction at 24 hours, 48 hours, 72 hours, or 1 week after cessation of treatment
Improvements in inflammatory biomarkers at 24 hours, 48 hours, 72 hours, or 1 week after cessation of treatment
Reduction in post-surgery opioid use during the acute phase (0-24 hours post-treatment) or the delayed phase (24-120 hours post-treatment) or both
Time to self-sufficient ambulation
Improvement in post-operative morbidity
Improvement in length of survival post-surgery

Dosing Regimen (for in-patient or out-patient setting):
Perioperative infusion starting about 15 minutes to 2 hours before the surgery, and lasting until about 24 or 48 hours after the surgery (preferably under telemetry monitoring)

### Example 15

This example sets forth a protocol for preventing and managing the migration of cancerous cells (i.e. metastasis) that occurs during and following invasive surgical procedures to remove cancerous tumors. The protocol is carried out at the same rates of intravenous infusion described in Example 14 and Table 2 in the weight and molar ratios of lidocaine to methylnaltrexone described in Example 14 and Table 3, for a total 27 combinations of dosing ranges and ratios. The dose of lidocaine and methylnaltrexone administered will always be below the maximum tolerated dose of each individual ingredient based on the risk to the patient’s cardiovascular system, particular the risk to cause cardiac arrhythmias and, for methylnaltrexone, the dose that either induces diarrhea or that treats opioid-induced constipation. In particular, the methylnaltrexone plasma concentration will be kept below 1400 ng/ml, and the lidocaine plasma concentration will always be kept below 5 mg/L.

Surgical Procedure (non-laparoscopic):
thoracic, orthopedic, and abdominal surgeries
tumor resection, particularly tumors of the breast and pancreas
osteosarcoma (limb sparing surgery, amputation, or rotationplasty)

Clinical Improvements:
Pain reduction at 24 hours, 48 hours, 72 hours, or 1 week after cessation of treatment
Improvements in inflammatory biomarkers at 24 hours, 48 hours, 72 hours, or 1 week after cessation of treatment
Reduction in post-surgery opioid use during the acute phase (0-24 hours post-treatment) or the delayed phase (24-120 hours post-treatment) or both
Improvement in post-operative morbidity
Improvement in length of survival post-surgery

Dosing Regimen (for in-patient or out-patient setting):
Perioperative infusion starting about 15 minutes to 2 hours before the surgery, and lasting until about 24 or 48 hours after the surgery (preferably under telemetry monitoring)

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of treating inflammation resulting from an invasive surgical procedure in a human in need thereof comprising administering to the human as an intravenous infusion a pharmaceutically acceptable composition comprising:
a) a therapeutically effective amount of lidocaine or a pharmaceutically acceptable salt thereof; and
b) a therapeutically effective amount of methylnaltrexone or a pharmaceutically acceptable salt thereof.

2. A method of inhibiting proliferation or metastasis of cancer cells following an invasive surgical procedure to remove a cancerous tumor in a human in need thereof comprising administering to the human as an intravenous infusion a pharmaceutically acceptable composition comprising:
a) a therapeutically effective amount of lidocaine or a pharmaceutically acceptable salt thereof; and
b) a therapeutically effective amount of methylnaltrexone or a pharmaceutically acceptable salt thereof.

3. A method of inhibiting Src tyrosine protein kinase phosphorylation at Tyr419 following an invasive surgical procedure in a human in need thereof comprising administering to the human as an intravenous infusion a pharmaceutically acceptable composition comprising:
a) a therapeutically effective amount of lidocaine or a pharmaceutically acceptable salt thereof; and
b) a therapeutically effective amount of methylnaltrexone or a pharmaceutically acceptable salt thereof.

4. A method of inhibiting cell signalling mediated by Src tyrosine protein kinase phosphorylation following an invasive surgical procedure in a human in need thereof comprising administering to the human as an intravenous infusion a pharmaceutically acceptable composition comprising:
a) a therapeutically effective amount of lidocaine or a pharmaceutically acceptable salt thereof; and
b) a therapeutically effective amount of methylnaltrexone or a pharmaceutically acceptable salt thereof.

5. A method of treating a disease mediated by Src tyrosine protein kinase phosphorylation following an invasive surgical procedure in a human in need thereof comprising administering to the human as an intravenous infusion a pharmaceutically acceptable composition comprising:
a) a therapeutically effective amount of lidocaine or a pharmaceutically acceptable salt thereof; and
b) a therapeutically effective amount of methylnaltrexone or a pharmaceutically acceptable salt thereof.

6. The method of claim 1, wherein the surgery is a non-laparoscopic tumor resection, preferably from the breast, pancreas, or osteosarcoma.

7. The method of claim 1, wherein the patient experiences an improvement in length of survival post-surgery or a reduction in post-operative morbidity.

8. The method of claim 2, wherein the surgery is a non-laparoscopic surgery selected from:
a) thoracic, orthopedic, and abdominal surgeries;
b) hemorrhoidectomies and bunionectomies;
c) hip arthroplasty, knee arthroplasty, and inguinal hernia repair; and
d) tumor resection, preferably from the breast, pancreas, or osteosarcoma.

9. The method of claim 2, wherein the patient experiences:
a) a reduction in pain at 24 hours, 48 hours, 72 hours, or 1 week after the treatment;
b) a reduction in post-surgery opioid use during the acute phase (0-24 hours post-treatment) or the delayed phase (24-120 hours post-treatment);
c) a reduction in time to self-sufficient ambulation; or
d) a reduction in post-operative morbidity.

10. The method of any of claims 1-9 comprising:
a) administering the composition as a continuous infusion before the procedure;
b) administering the composition as a continuous infusion during the procedure;
c) administering the composition as a continuous infusion after the procedure; or
d) any combination of (a)-(c).

11. The method of any of claims 1-9, comprising administering the composition during the perioperative period.

12. The method of claim 11 wherein the lidocaine or pharmaceutically acceptable salt thereof is administered as lidocaine hydrochloride.

13. The method of claim 11 wherein the lidocaine or pharmaceutically acceptable salt thereof is administered in a daily amount of from 10 to 3000 mg.

14. The method of claim 11 wherein the methylnaltrexone is administered as methylnaltrexone bromide.

15. The method of claim 11, wherein the methylnaltrexone or pharmaceutically acceptable salt thereof is administered in a daily amount of from 0.2 to 175 mg.

16. The method of claim 11 wherein the lidocaine or pharmaceutically acceptable salt thereof is administered in a daily amount of from 10 to 3000 mg and the methylnaltrexone or pharmaceutically acceptable salt thereof is administered in a daily amount of from 0.2 to 175 mg.

17. The method of claim 11 wherein the lidocaine or pharmaceutically acceptable salt thereof is administered at a rate of from 10 to 45 mg/kg/day, and the methylnaltrexone or pharmaceutically acceptable salt thereof is administered at a rate of from 0.2 to 2 mg/kg/day.

18. The method of claim 11 wherein the lidocaine or pharmaceutically acceptable salt thereof is administered at a rate of from 15 to 35 mg/kg/day, and the methylnaltrexone or pharmaceutically acceptable salt thereof is administered at a rate of from 0.25 to 1.75 mg/kg/day.

19. The method of claim 11 wherein the lidocaine or pharmaceutically acceptable salt thereof is administered at a rate of from 20 to 30 mg/kg/day, and the methylnaltrexone or pharmaceutically acceptable salt thereof is administered at a rate of from 0.35 to 1.5 mg/kg/day.

20. The method of claim 16 wherein the methylnaltrexone or pharmaceutically acceptable salt thereof and lidocaine or pharmaceutically acceptable salt thereof are administered at a weight ratio of from 1:10 to 1:125.

21. The method of claim 17 wherein the methylnaltrexone or pharmaceutically acceptable salt thereof and lidocaine or pharmaceutically acceptable salt thereof are administered at a weight ratio of from 1:10 to 1:125.

22. The method of claim 18 wherein the methylnaltrexone or pharmaceutically acceptable salt thereof and lidocaine or pharmaceutically acceptable salt thereof are administered at a weight ratio of from 1:10 to 1:125.

23. The method of claim 19 wherein the methylnaltrexone or pharmaceutically acceptable salt thereof and lidocaine or pharmaceutically acceptable salt thereof are administered at a weight ratio of from 1:10 to 1:125.

24. The method of claim 11 wherein the patient is suffering from a cancerous tumor that relies on angiogenic processes.

25. The method of claim 11 wherein the patient is suffering from a tumor of the pancreas, kidney, liver, lung, colon, rectum, breast, bladder, or bone.

26. The method of any of the foregoing claims, wherein the dose of methylnaltrexone increases plasma methylnaltrexone concentrations to no more than 1400 ng/mL, and the dose of lidocaine increases the plasma lidocaine concentration to no more than 5 mg/L.

27. An intravenous pharmaceutical composition in the form of a sterile liquid or powder comprising:
a) a therapeutically effective amount of lidocaine or a pharmaceutically acceptable salt thereof;
b) a therapeutically effective amount of methylnaltrexone or a pharmaceutically acceptable salt thereof; and
c) one or more pharmaceutically acceptable carriers.

28. The composition of claim 27 in the form of a unit dose or multi-dose sterile liquid or powder for intravenous administration.

29. The composition of claim 27 wherein the lidocaine is in the form of lidocaine hydrochloride.

30. The composition of claim 27 wherein the methylnaltrexone is present as methylnaltrexone bromide.

31. The composition of claim 27 wherein the methylnaltrexone or pharmaceutically acceptable salt thereof and lidocaine or pharmaceutically acceptable salt thereof are present at a weight ratio of from 1:10 to 1:125.

32. The composition of claim 27 wherein the methylnaltrexone or pharmaceutically acceptable salt thereof and lidocaine or pharmaceutically acceptable salt thereof are present at a weight ratio of from 1:20 to 1:100.

33. The composition of claim 27 wherein the methylnaltrexone or pharmaceutically acceptable salt thereof and lidocaine or pharmaceutically acceptable salt thereof are present at a weight ratio of from 1:30 to 1:75.
